(19) United States
(12) Patent Application Publication
(10) Pub. No.: US 2011/0124091 A1
(43) Pub. Date: May 26, 2011

(54) INDUSTRIALIZED ALGAE CULTURING METHOD AND SYSTEM THEREOF

(76) Inventors: Chao-Hui Lu, Hsin Chu City (TW); Hsing-Hong Lu, Hsin Chu City (TW)

(21) Appl. No.: 12/624,642

(22) Filed: Nov. 24, 2009

(51) Int. Cl. C12N 1/12; C12M 1/00

(52) U.S. Cl. 435/257.1; 435/292.1

(57) ABSTRACT

An industrialized algae culturing method is provided, which comprises the following steps of: placing algae and a culture solution into a pipeline photosynthesis unit for photosynthesis; introducing the photosynthesized algae and the culture solution into a gas intake/venting unit by means of a powered liquid transport unit to remove oxygen from and replenish carbon dioxide into the culture solution; and introducing the photosynthesized algae and the culture solution into the pipeline photosynthesis unit for recycling therein. the gas intake/venting unit has a first sealing member and a second sealing member, and the first sealing member and the second sealing member keep the gas intake/venting unit sealed off the external environment during a process of gas intake or venting, thereby keeping the culture solution clean and improving the quality of the algae. Furthermore, the present invention also provides an industrialized algae culturing system.

### BACKGROUND OF THE INVENTION

1. Field of the Invention

The present invention relates to an industrialized algae culturing method and a system thereof, and more particularly, to an industrialized algae culturing method and a system thereof which enable algae to carry on photosynthesis cyclically by means of a pipeline culturing system.

2. Description of Related Art

Algae, such as Spirulina, Haematococcus Pluvialis Flotow or Botryocladia Leptopoda, are known to be rich in proteins, minerals, vitamins, enzymes, antioxidants, astaxanthins and the like nutrient ingredients that are beneficial to people's health, and have been widely recommended as a kind of health food in recent years. Even further, biodiesel can now be extracted from algae for use as a kind of energy source. Through a photosynthesis reaction system, an algae culture solution can carry on photosynthesize to produce nutrition required for alga cells to grow so that blue-green algae can be cultured in mass. Besides, oxygen produced in the photosynthesis process is also exhausted into the culture solution to increase the oxygen content thereof.

A conventional photosynthesis reaction system for algae is a big outdoor culturing pool containing a culture solution to carry on photosynthesis therein. However, the big outdoor culturing pool occupies a large land area, consumes much energy, and its use is restricted by the weather; and especially, the algae obtained are susceptible to deterioration in quality due to pollution. All of this causes a lot of trouble to the manufacturers.

Accordingly, in view of the aforesaid shortcomings, the present inventor has made great research efforts based on application of theories, and finally proposed the present invention that features a rational design and can effectively overcome the aforesaid shortcomings.

### SUMMARY OF THE INVENTION

An objective of the present invention is to provide an industrialized algae culturing method and a system thereof, which are intended to increase yield of harvestable algae and avoid pollution of the culture solution by the external environment, thereby improving quality of the algae.

To achieve the above-mentioned objectives, the present invention provides an industrialized algae culturing method, comprising the following steps of: placing algae and a culture solution into a pipeline photosynthesis unit so that the algae carry on photosynthesis to produce photosynthesized algae and oxygen; introducing the photosynthesized algae and the culture solution into a gas intake/venting unit by means of a powered liquid transport unit, wherein the gas intake/venting unit is adapted to remove the oxygen from and replenish carbon dioxide into the culture solution, and the gas intake/venting unit comprises a first sealing member and a second sealing member, and wherein the first sealing member and the second sealing member keep the gas intake/venting unit sealed off the external environment during a process of gas intake or venting; and introducing the photosynthesized algae and the culture solution into the pipeline photosynthesis unit for recycling therein.

The present invention also provides an industrialized algae culturing system, comprising: a pipeline photosynthesis unit, comprising a transparent piping that contains algae and a culture solution therein; a powered liquid transport unit, being connected with the pipeline photosynthesis unit at the upstream thereof and being used to pressurized the culture solution in the transparent piping; a gas intake/venting unit, being connected with the powered liquid transport unit at the upstream thereof and comprising a closed liquid collecting cylinder, a venting pipe and a gas feeding pipe, wherein the venting pipe keeps the closed liquid collecting cylinder sealed off the external environment by means of a first sealing member, and the gas feeding pipe keeps the closed liquid collecting cylinder sealed off the external environment by means of a second sealing member; and a communicating pipe unit, communicating with the gas intake/venting unit at the upstream thereof and with the pipeline photosynthesis unit at the downstream thereof.

The present invention provides the following benefits: as the first sealing member and the second sealing member function by keeping the gas intake/venting unit sealed off the external environment during the process of gas intake and venting of the gas intake/venting unit, pollution of the culture solution by the external environment is avoided and, consequently, the culture solution is kept clean, which is favorable for growth and reproduction of the algae.

For further understanding of the features and technical details of the present invention, reference will be made to the detailed descriptions hereinbelow and the attached drawings; however, the attached drawings are merely provided for purpose of reference and illustration, but not to limit the scope of the present invention.

### BRIEF DESCRIPTION OF THE DRAWINGS

FIG. 1 is a flowchart of an industrialized algae culturing method according to the present invention;

FIG. 2 is a schematic view of an industrialized algae culturing system according to the present invention;

FIG. 2A is another schematic view of the industrialized algae culturing system according to the present invention;

FIG. 3 is a schematic view of a cell division unit according to the present invention;

FIG. 4 is another schematic view of the industrialized algae culturing system according to the present invention;

FIG. 5 is a flowchart illustrating steps of the culturing method according to the present invention;

FIG. 6 is yet another schematic view of the industrialized algae culturing system according to the present invention;

FIG. 7A is a schematic view of a varied embodiment of the industrialized algae culturing system according to the present invention; and

FIG. 7B is a schematic view of another varied embodiment of the industrialized algae culturing system according to the present invention.

### DETAILED DESCRIPTION OF THE PREFERRED EMBODIMENTS

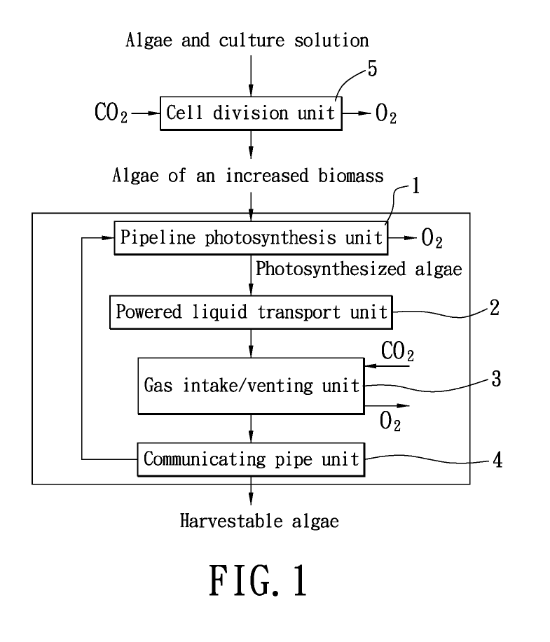

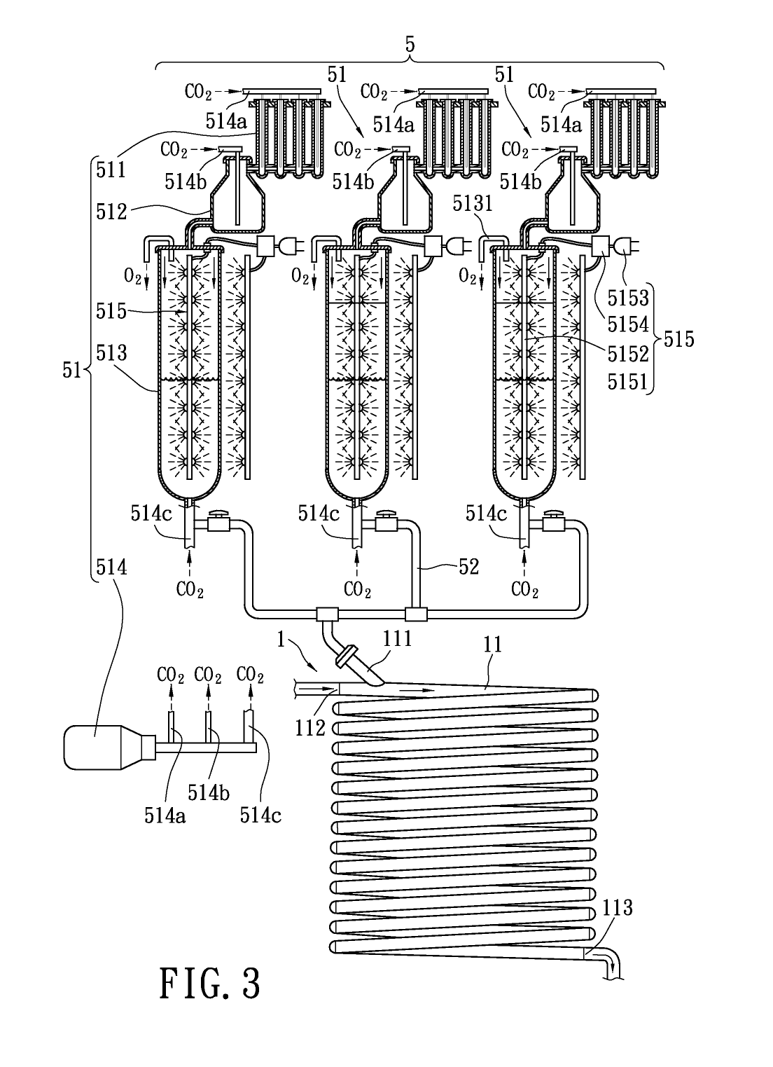

Referring now to FIGS. 1, 2 and 3, an industrialized algae culturing system according to the present invention is shown therein. The industrialized algae culturing system comprises a pipeline photosynthesis unit 1, a powered liquid transport unit 2, a gas intake/venting unit 3, a communicating pipe unit 4 and a cell division unit 5. With this arrangement, algae and a culture solution are initially placed into the cell division unit 5 to produce the algae with biomass increased, which are then injected into the pipeline photosynthesis unit 1 for photosynthesis to get photosynthesized algae and oxygen. Then, by means of the powered liquid transport unit 2, the photosynthesized algae and the culturing solution are introduced into the gas intake/venting unit 3 to remove oxygen from and replenish carbon dioxide into the culture solution. Finally, the photosynthesized algae and the culturing solution are introduced into the communicating pipe unit 4 to physiologically regulate the algae for a period of time, and the photosynthesized algae and the culturing solution are cyclically introduced into the pipeline photosynthesis unit 1 to allow the algae to grow and reproduce gradually into harvestable algae. Eventually, the harvestable algae are harvested.

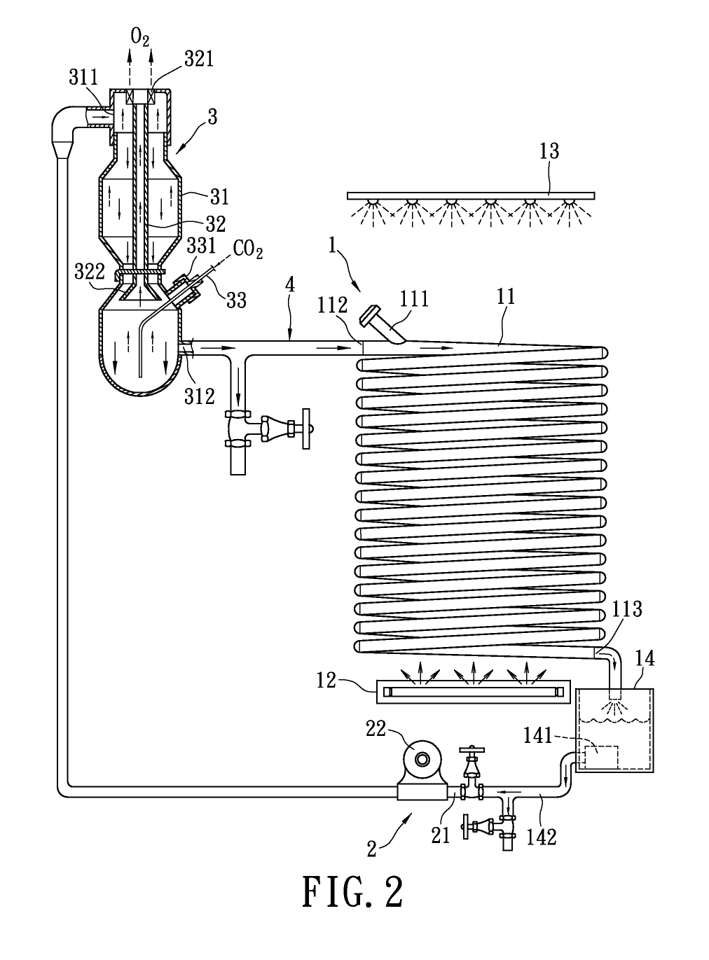

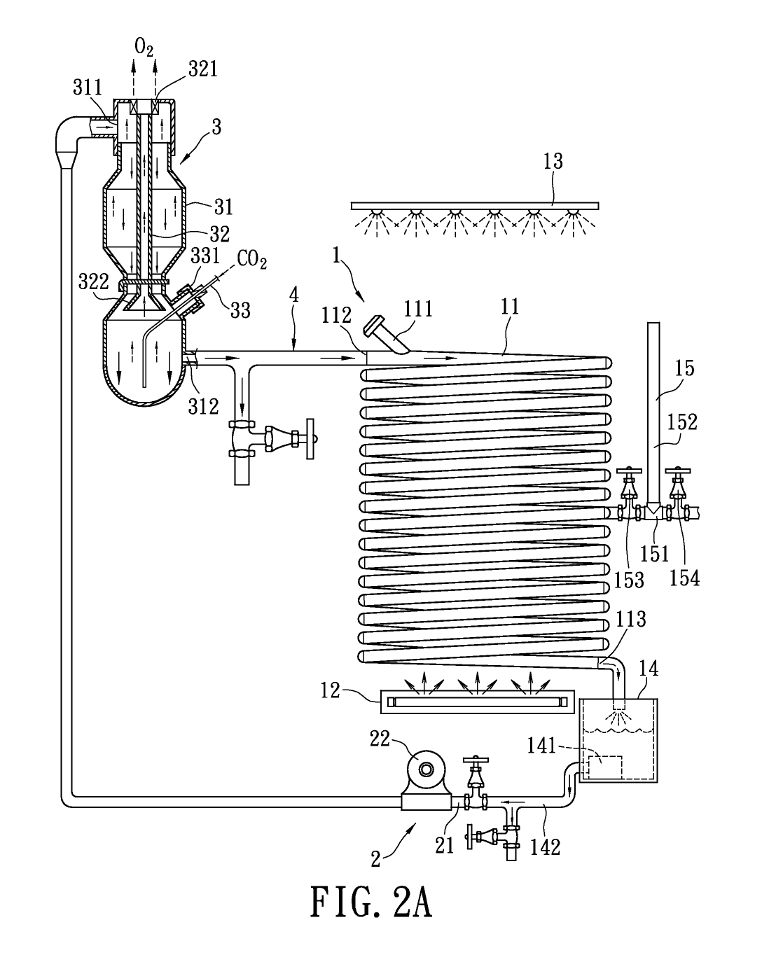

The pipeline photosynthesis unit 1 (as shown in FIG. 2) comprises a transparent piping 11, a light supplement unit 12, a water sprayer unit 13 and a temperature controlling sink 14. The transparent piping 11 receives illumination of light from a fluorescent lamp, a light emitting diode (LED) lamp or the sunlight. An algae injection port 111 and a photosynthesis water inlet 112 are disposed at one end of the transparent piping 11, while a photosynthesis water outlet pipe 113 is disposed at the other end of the transparent piping 11. Thus, via the algae injection port 111, the prepared algae and the culture solution are injected into the transparent piping 11 to flow therethrough. By absorbing the light illumination and carbon dioxide contained in the culture solution, the algae carry on photosynthesis to grow and reproduce and, meanwhile, produce oxygen. Then, the photosynthesized algae and the culture solution flow out of the transparent piping 11 via the photosynthesis water outlet pipe 113.

The light supplement unit 12, which is a fluorescent lamp or an LED lamp, is disposed at an appropriate location near the transparent piping 11; for example, a distance between the transparent piping 11 and the light supplement unit 12 or an illumination angle of light may be adjusted appropriately. Here, the light supplement unit 12 of the present invention is disposed beneath the transparent piping 11, although it is not merely limited thereto. Light emitted from the light supplement unit 12 illuminates the transparent piping 11 and enhances the light intensity received by the transparent piping 11, so as to promote growth and reproduction of the algae by providing an adequate light intensity necessary for photosynthesis of the algae. In this way, the intensity and illumination angle of light received by the algae can be adjusted according to various growing environments suitable for different algae.

The water sprayer unit 13, which is a manual or a sensor-controlled water sprayer, is disposed at an appropriate location near the transparent piping 11; here, the water sprayer unit 13 of the present invention is disposed above the transparent piping 11, although it is not limited thereto. By spraying water onto the transparent piping 11, the water sprayer unit 13 functions by decreasing the temperature of the culture solution within the transparent piping 11. This helps to avoid a too high temperature of the culture solution due to the light illumination, thereby providing a suitable environmental temperature for growth and reproduction of the algae. In this way, temperature of the culture solution is controlled according to various growing environments suitable for different algae.

The photosynthesis water outlet pipe 113 is connected to the temperature controlling sink 14 to introduce the photosynthesized algae and culture solution into the temperature-controlling sink 14. The temperature controlling sink 14 is provided with a temperature controlling unit 141 and a temperature controlling sink water outlet pipe 142. The temperature controlling unit 141, which is a heater or a cooler, is configured to control temperature of the culture solution by heating or cooling the temperature controlling sink 14. The temperature controlling sink water outlet pipe 142 is used to introduce the photosynthesized algae and culture solution into the powered liquid transport unit 2. In this way, temperature of the culture solution in the temperature controlling sink 14 can be controlled by the temperature controlling unit 141 to facilitate growth and reproduction of the algae. Especially, in case of a too high or too low temperature unsuitable for growth and reproduction of the algae, the temperature controlling sink 14 will operate to increase or decrease the temperature of the culture solution.

The powered liquid transport unit 2 has a powered liquid transport connecting pipe 21 and a pressurized liquid pump 22. One end of the powered liquid transport connecting pipe 21 can communicate with the water pipe of the temperature controlling sink 14, while the other end thereof is connected to the gas intake/venting unit 3. The pressurized liquid pump 22 is adapted to pressurize the culture solution in the powered liquid transport connecting pipe 21 so as to force the pressurized culture solution into the gas intake/venting unit 3.

The gas intake/venting unit 3 is a hollow cylindrical part comprising a closed liquid collecting cylinder 31, a venting pipe 32 and a gas feeding pipe 33. The closed liquid collecting cylinder 31 has a gas venting/water inlet port 311 disposed at the top thereof, and a gas venting/water outlet pipe 312 disposed at the bottom thereof. One end of the venting pipe 32 is disposed at the top of the closed liquid collecting cylinder 31 and a first sealing member 321 is used to isolate the closed liquid collecting cylinder 31 out of the external environment so that foreign matters from the external environment is prevented from entering the closed liquid collecting cylinder 31. The other end of the venting pipe 32 extends towards the bottom of the closed liquid collecting cylinder 31 to form an enlarged portion 322. The gas feeding pipe 33 is disposed on a sidewall of the closed liquid collecting cylinder 31 and a second sealing member 331 is used to isolate the closed liquid collecting cylinder 31 out of the external environment so that foreign matters from the external environment is prevented from entering the closed liquid collecting cylinder 31. However, each of the first sealing member 321 and the second sealing member 331 is a filter screen or an air valve, which allows oxygen or carbon dioxide to pass therethrough while preventing entry of foreign matters from the external environment into the closed liquid collecting cylinder 31. Thus, the culture solution transported by the powered liquid transport unit 2 flows through the gas venting/water inlet port 311 into the closed liquid collecting cylinder 31 and strikes the enlarged portion 322 to form diffused splashes, which are then collected at the bottom of the closed liquid collecting cylinder 31. Meanwhile, oxygen in the culture solution is exhausted out of the closed liquid collecting cylinder 31 via the venting pipe 32. Furthermore, via the gas feeding pipe 33, carbon dioxide is introduced into the culture solution contained at the bottom of the closed liquid collecting cylinder 31 to supply carbon dioxide required for photosynthesis of the algae, thereby improving capability of the culture solution to carry on photosynthesis again. Accordingly, the gas intake/venting unit 3 vents oxygen from the culture solution via the venting pipe 32 and replenishes carbon dioxide into the culture solution via the gas feeding pipe 33. During the process of gas intake and venting, the first and the second sealing members 321, 331 keep the gas intake/venting unit 3 sealed off the external environment, so pollution of the culture solution by the external environment is prevented and, consequently, the culture solution is kept clean, which is favorable for growth and reproduction of the algae.

The communicating pipe unit 4 is a closed piping, one end of which communicates with and may be directly connected to the gas venting/water outlet pipe 312 while the other end is connected to the photosynthesis water inlet 112. Via the communicating pipe unit 4, the culture solution is introduced into the transparent piping 11 to have the algae carry on photosynthesis again for growth and reproduction.

The cell division unit 5 is located upstream of the pipeline photosynthesis unit 1. The algae are firstly placed into the cell division unit 5 to accelerate cell division of the algae, resulting in an adequate number of algae cells and the algae with biomass increased. The cell division unit 5 comprises a plurality of consecutive and sealed containers 51, each of which further comprises a plurality of small-sized test tubes 511, a medium-sized beaker 512 and an air-lift photosynthesis unit 513. The medium-sized beaker 512 is connected at the downstream of the small-sized test tubes 511 and, in turn, the air-lift photosynthesis unit 513 is connected at the downstream of the medium-sized beaker 512 to form a consecutive and sealed container 51 which is consecutive in nature and sealed. With this arrangement, the algae and the culture solution are placed into each of the small-sized test tubes 511 for cell division of the algae. Once cells of the algae divide to a certain number, the algae and the culture solution contained in each of the small-sized test tubes 511 are introduced into the medium-sized beaker 512 to allow for further cell division of the algae. Finally, the algae and the culture solution are introduced from the medium-sized beaker 512 into the air-lift photosynthesis unit 513 where the algae are illuminated by a light source module 515 to carry on photosynthesis. This helps to accelerate cell division of the algae in each of the consecutive and sealed containers 51, resulting in a great mass of algae. The light source module 515 comprises a first light source 5151, a second light source 5152, a power supply 5153 and a power controlling unit 5154. Via the power controlling unit 5154, the power supply 5153 is electrically connected to the first light source 5151 and the second light source 5152 to supply power necessary for illumination; meanwhile, illumination intensity of the first light source 5151 and the second light source 5152 can be adjusted to control the temperature of the algae and the culture solution within the air-lift photosynthesis unit 513. The first light source 5151 is disposed outside the air-lift photosynthesis unit 513, while the second light source 5152 is disposed inside the air-lift photosynthesis unit 513 so that the algae can receive enough light illumination for photosynthesis to increase the biomass yield. Additionally, the blowing device 514 comprises a first piping 514a, a second piping 514b and a third piping 514c. The first piping 514a is connected to each of the small-sized test tubes 511, the second piping 514b is connected to each of the medium-sized beakers 512, and the third piping 514c is connected to each of the air-lift photosynthesis units 513. Via the first piping 514a, the second piping 514b and the third piping 514c, carbon dioxide is blown by the blowing device 514 into the small-sized test tubes 511, the medium-sized tubes 512 and the air-lift photosynthesis units 513, respectively, to induce flow of the algae. This results in even distribution of the algae in the culture solution, which facilitates growth and cell division of the algae to result in an increased biomass for algae. Oxygen produced from the photosynthesis is vented to the external environment via the venting pipe 5131, while the algae of increased biomass and the culture solution are introduced from the algae injection port 111 through a manifold piping 52 into the transparent piping 11 for photosynthesis. Therefore, the consecutive and sealed container 51 is favorable for cell division of the algae to produce the algae with biomass increased in the culture solution that can carry on photosynthesize for growth and reproduction, thereby increasing yield of the algae.

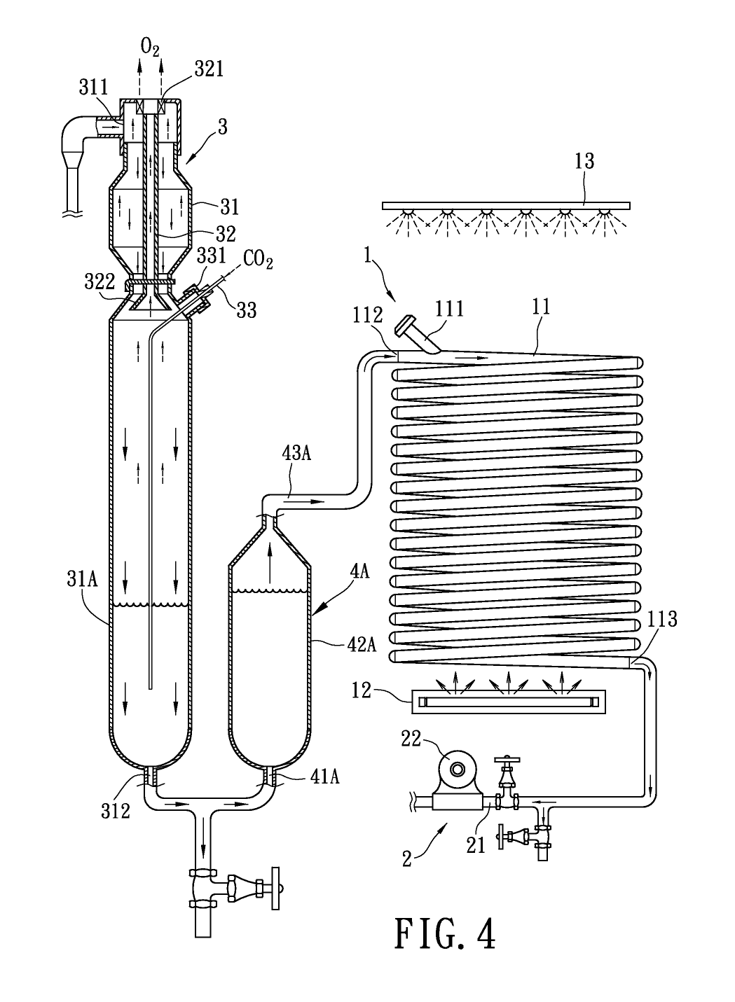

Furthermore, the gas intake/venting unit 3 and the communicating pipe unit 4 may be varied in design. Referring to FIG. 4, it differs in that, the gas intake/venting unit 3 may use a longer closed liquid collecting cylinder 31A and is arranged side by side to the communicating pipe unit 4A, in which the communicating pipe unit 4A has a communicating water inlet 41A, an enlarged communicating pipe 42A and a communicating water outlet pipe 43A. The bottom of the enlarged communicating pipe 42A communicates with the communicating water inlet 41A, the top of the enlarged communicating pipe 42A communicates with the communicating water outlet pipe 43A, the communicating water inlet 41A is connected to the gas venting/water outlet pipe 312, and the communicating water outlet pipe 43A is connect with the photosynthesis water inlet 112. Thus, when the culture solution collected in the closed liquid collecting cylinder 31A reaches a liquid level higher than that of the enlarged communicating pipe 42A, the culture solution will be forced by the pressure, via the communicating water outlet pipe 43A, into the transparent piping 11 where the algae will early on photosynthesis anew for growth and reproduction. As both the longer closed liquid collecting cylinder 31A and the enlarged communicating pipe 42A of an increased diameter slow down the flow rate of the culture solution, it takes a longer time for the culture solution to flow from the communicating pipe unit 4A into the transparent piping 11. This allows the algae to be physiologically regulated for an enough period of time to eliminate physiologic harm caused by the powered liquid transport unit 2 and the gas intake/venting unit 3 to the algae, thereby obtaining algae of the optimal quality. In this way, by using the communicating pipe unit 4A to slow down the flow rate of the culture solution, the purpose to perform physiologic regulation for the algae is accomplished. Furthermore, owing to the differential pressure arising from difference in liquid levels of the culture solution in the closed liquid collecting cylinder 31A and in the communicating pipe unit 4A, the culture solution is cyclically forced into the pipeline photosynthesis unit 1 where they will carry on photosynthesis anew for growth and reproduction.

Further, referring back to FIG. 2A, for purpose of mass production, the transparent piping 11 can typically be enlarged in either the diameter or the length to increase the yield. However, a too long piping 11 would cause oxygen content to be too high to slow down the growth rate. For this reason, a ventilation valve assembly 15 may be connected at a middle section of the transparent piping 11 functions by increasing the yield. The ventilation valve assembly 15 has a communicating water pipe 151 connected with the transparent piping 11, a venting pipe 152 connected with the communicating water pipe 151 and having a height beyond that of the transparent piping 11, a switching valve 153 for switching the communicating water pipe 151, and a drain valve 154. Hence, when the switching valve 153 is opened, the culture solution will flow to the venting pipe 152, and because the venting pipe 152 has a height beyond that of the transparent piping 11, the culture solution will not overflow and oxygen can be vented outwards via the venting pipe 152 successfully. Alternatively, other facilities may be additionally provided to make it easier to vent oxygen to the outside. For example, the ventilation valve assembly 15 may be instead by a gas intake/venting unit such as the gas intake/venting unit 3 shown in FIG. 2. By opening the drain valve 154, necrotic algae can be drained off.

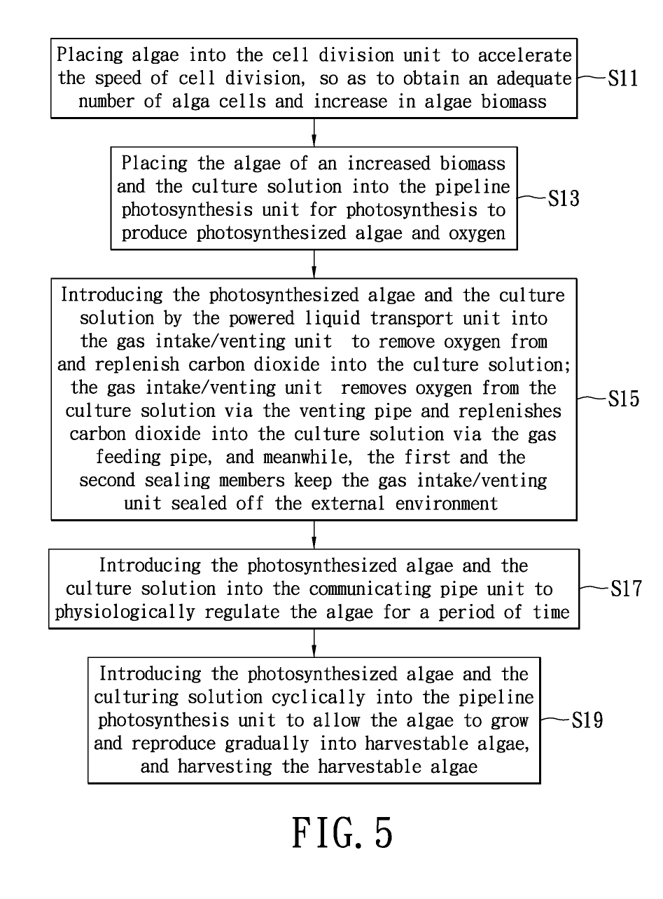

Referring to FIGS. 2 to 5, the present invention further provides an industrialized algae culturing method, which comprises the following steps of:

In step S11, algae are placed into the cell division unit 5 to accelerate the speed of cell division, in order to obtain an adequate number of alga cells and increase in algae biomass. The algae and the culture solution are placed into a plurality of small-sized test tubes 511 respectively to allow for cell division of the algae; then, the algae and the culture solution in each of the small-sized test tubes 511 are introduced into the medium-sized beaker 512 to allow for further cell division of the algae; and finally, the algae and the culture solution in the medium-sized beaker 512 are introduced into the air-lift photosynthesis unit 513. In the air-lift photosynthesis unit 513, the algae are exposed to illumination from the light source module 515 for photosynthesis so as to accelerate cell division of the algae to obtain a great mass of algae. Moreover, by means of the blowing device 514, gas is blown into the air-lift photosynthesis unit 513 to induce flow of the massive algae. This results in even distribution of the algae in the culture solution, which facilitates growth and cell division of the algae to result in an increased biomass of algae.

In step S13, the algae of an increased biomass and the culture solution may be placed into the pipeline photosynthesis unit 1 for photosynthesis to produce photosynthesized algae and oxygen.

In step S15, the photosynthesized algae and the culture solution are introduced by the powered liquid transport unit 2 into the gas intake/venting unit 3 to remove oxygen from and replenish carbon dioxide into the culture solution. The gas intake/venting unit 3 removes oxygen from the culture solution via the venting pipe 32 and replenishes carbon dioxide into the culture solution via the gas feeding pipe 33. Meanwhile, the first and the second sealing members 321, 331 keep the gas intake/venting unit 3 sealed off the external environment.

In step S17, the photosynthesized algae and the culture solution are introduced into the communicating pipe unit 4 to physiologically regulate the algae for a period of time. Then, by virtue of difference in liquid levels of the culture solution in the gas intake/venting unit 3 and that in the communicating pipe unit 4, the culture solution are forced into the transparent piping 11 cyclically.

In step S19, the photosynthesized algae and the culturing solution are cyclically introduced into the pipeline photosynthesis unit 1 to allow the algae to grow and reproduce gradually into harvestable algae. Eventually, the harvestable algae are harvested.

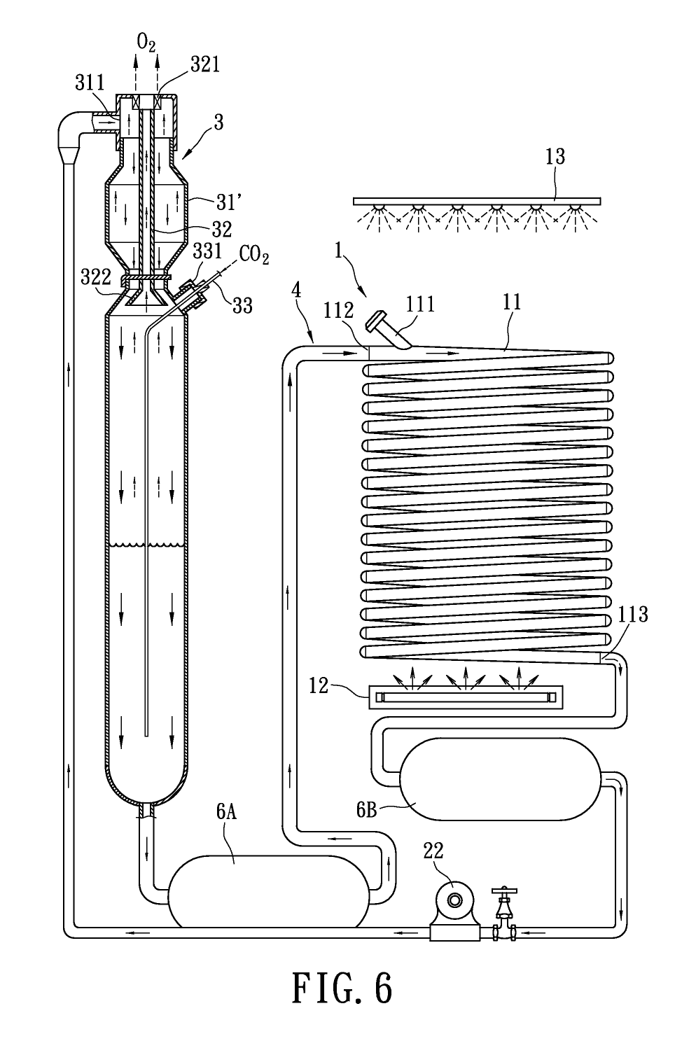

Referring next to FIG. 6, in this embodiment, in order to further increase the production capacity of algae significantly, the closed liquid collecting cylinder 31′ of the gas intake/venting unit 3 may be lengthened at the bottom to nearly reach the ground (approximately 2-3 m from the ground), and may further communicate with at least a transparent first liquid storage pipe 6A, which is disposed on the ground, at the bottom. The first liquid storage pipe 6A may be sized to have a length of about 3-4 m and a diameter of about 30 cm. In this embodiment, the communicating pipe unit 4 communicates with the gas intake/venting unit 3 at the upstream via the first liquid storage pipe 6A, and communicates with the transparent piping 11 directly at the downstream. Because of the large size thereof, the first liquid storage pipe 6A is placed flat on the ground so as to be fixed easily. However, a second liquid storage pipe 6B which has the same dimensions as the first liquid storage pipe 6A may also be disposed to communicate with the end of the transparent piping 11, and the second liquid storage pipe 6B is connected to the powered liquid transport unit 2 in order to communicate with the transparent piping 11. However, dimensions of the liquid storage pipes are not merely limited thereto, but may be adjusted appropriately depending on design requirements in practical production. With the varied design of this embodiment, the lengthened closed liquid collecting cylinder 31′, the first liquid storage pipe 6A and the second liquid storage pipe 6B can give rise to a further increase of about 3-40 tons in capacity and, through photosynthesis, also give rise to an increase in biomass. This is particularly beneficial to increase in biomass of such algae as Haematococcus Pluvialis Flotow, Botryocladia Leptopoda or the like and to make improvement in the oxygen content.

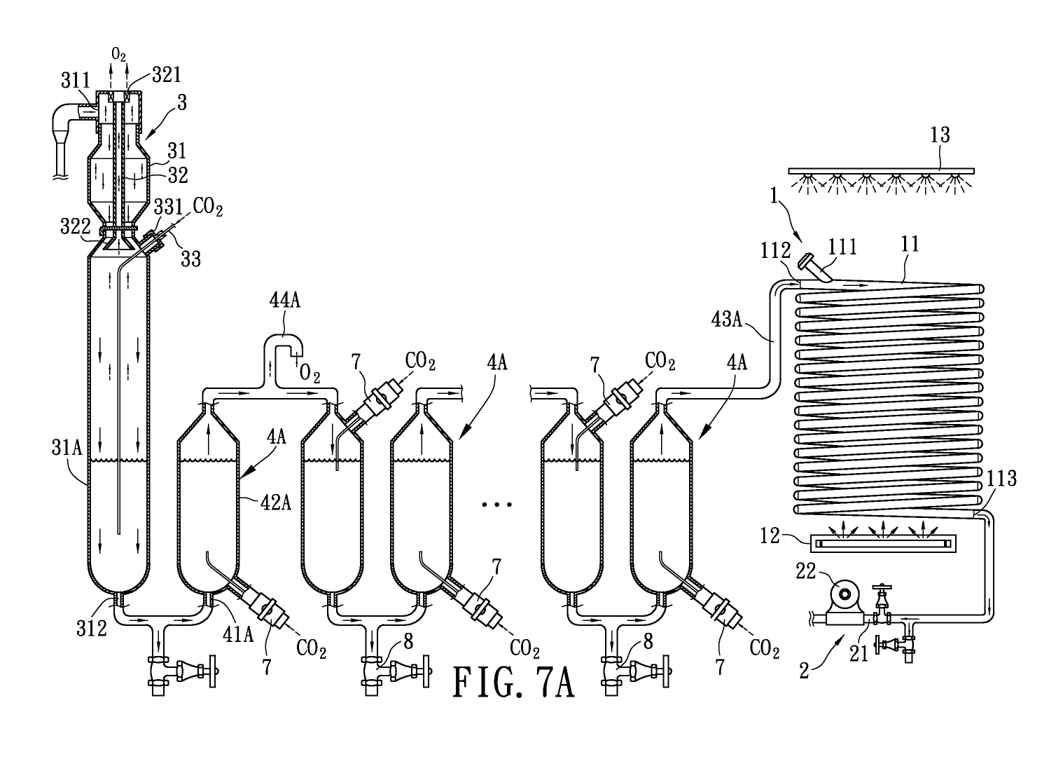

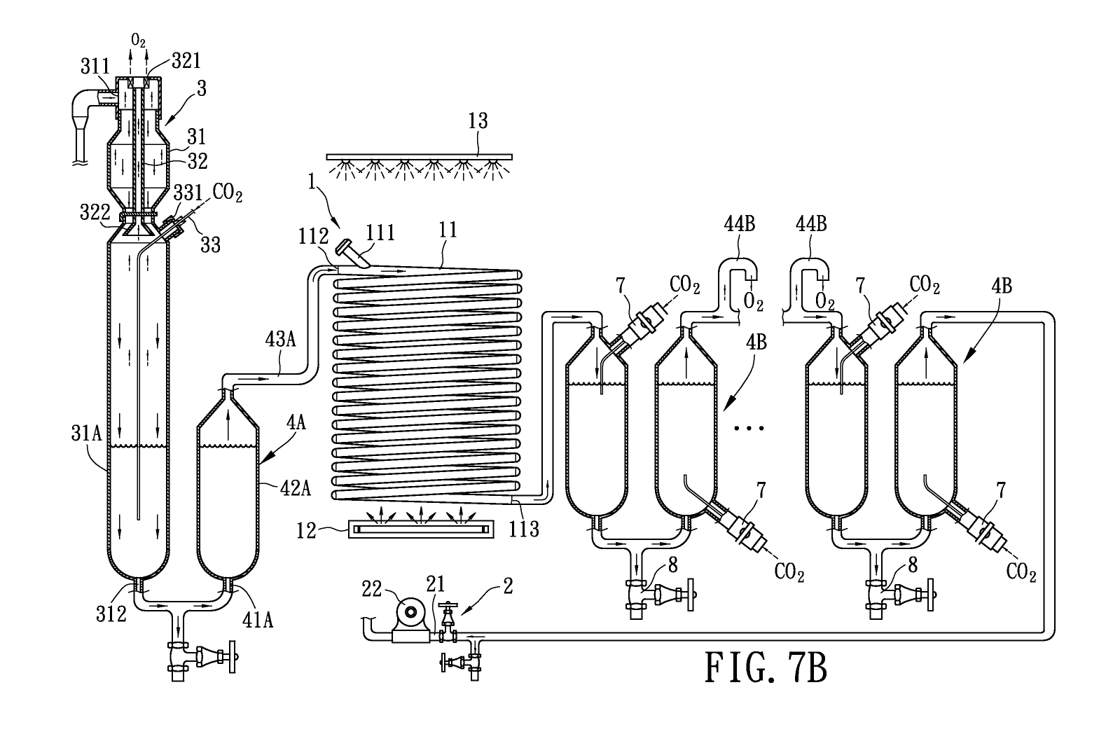

Referring next to FIG. 7A, in this varied embodiment of the present invention, the number of communicating pipe units 4A connected to the bottom of the gas intake/venting unit 3 and the transparent piping 11 may be varied; i.e., a plurality of communicating pipe units 4A that are in continuous flow communication, each of which can be an enlarged communicating pipe 42A, may be provided to connect with the bottom of the gas intake/venting unit 3 and the transparent piping 11. A switching valve assembly 8 is connected to the bottom of every two of the communicating pipe units 4A, each of the communicating pipe units 4A is connected with a gas pumping device 7, and a venting port 44 that is bent and open downwards may be provided at the top of every two of the communicating pipe units 4A in order to vent the gas (oxygen) and prevent foreign matters from entering the communicating pipe units 4A. The gas pumping devices 7 may be installed along the flowing direction of the algae; i.e., if the algae enters a communicating pipe unit 4A from the bottom thereof, the gas pumping device 7 may be installed at the bottom of the communicating pipe unit 4A, and if the algae enters another communicating pipe unit 4A from the top thereof, a corresponding gas pumping device 7 may be installed at the top of the another communicating pipe unit 4A. By use of the communicating pipe units 4A that are in continuous flow communication, the algae introduced from the gas intake/venting unit 3 can be physiologically regulated on a continuous basis for a period of time. By use of the gas pumping device 7, carbon dioxide is filled into the communicating pipe units 4A to induce flow of the massive algae so that the algae are evenly distributed in the culture solution, which is favorable for growth and cell division of the algae to increase the biomass. The switching valve assembly 8 is used to discharge the grown algae that have circulated through the whole system for harvesting. As compared to the above embodiment, the communicating pipe units 4A of this embodiment allows for harvesting everyday or every other day, and the continuous harvesting in massive quantities can continue without having to stop the system until cleaning of the whole system becomes necessary.

Referring to FIG. 7B, this embodiment differs from that of FIG. 7A in that, at an end of the transparent piping 11 are connected a plurality of communicating pipe units 4B that are in continuous flow communication. The algae and the culture solution from the pipeline photosynthesis unit 1 are firstly introduced into the communicating pipe units 4B that are in continuous flow communication where the algae grow and divide continuously for a period of time and then, through the powered liquid transport unit 2 which communicates with the communicating pipe units 4B, are introduced into the gas intake/venting unit 3. Besides, just as in the above embodiment, each of the communicating pipe units 4B is connected with a gas pumping device 7 and a venting port 44 that is bent and open downwards, and a switching valve assembly 8 is connected to the bottom of every two of the communicating pipe units 4B. This embodiment and the above embodiments are provided for use with different algae species, and the number of elements also varies depending on different algae species. By applying either one of these two embodiments, it can allow for harvesting every day or every other day.

In summary, the present invention has the following features:

(1) During the process of gas intake and venting of the gas intake/venting unit 3, the first and the second sealing members 321, 331 keep the gas intake/venting unit 3 sealed off the external environment, so pollution of the culture solution by the external environment is prevented and, consequently, the culture solution is kept clean, which is favorable for growth and reproduction of the algae.

(2) Before being placed into the pipeline photosynthesis unit 1, the algae are firstly placed into the cell division unit 5 to accelerate cell division of the algae, resulting in an adequate number of algae cells and increasing in algae biomass; then the algae of an increased biomass are placed into the pipeline photosynthesis unit 1 where the algae carry on photosynthesis cyclically to grow and reproduce into harvestable algae. Hence, through the two stages of, namely, cell division as well as growth and reproduction, yield of the algae is increased.

(3) As the consecutive and sealed container 51 comprises containers of three different sizes, i.e., the small-sized test tubes 511, the medium-sized beaker 512 and the air-lift photosynthesis unit 513, the algae that flow and carry on photosynthesis in the consecutive and sealed container 51 can experience staged cell division, which helps to accelerate the speed of cell division of the algae.

(4) By means of the blowing device 514, carbon dioxide is blown into the air-lift photosynthesis unit 513 to induce flow of the massive algae. This results in even distribution of the algae in the culture solution, which facilitates growth and cell division of the algae to result in an increased biomass of algae.

The above descriptions are only provided to illustrate the preferred embodiments of the present invention, but not to limit the scope of the present invention. Accordingly, various equivalent changes, alternations or modifications based on the claims of present invention are all consequently viewed as being embraced by the scope of the present invention.

What is claimed is:

1. An industrialized algae culturing method, comprising the following steps of:
placing algae and a culture solution into a pipeline photosynthesis unit so that the algae carry on photosynthesis to produce photosynthesized algae and oxygen;
introducing the photosynthesized algae and the culture solution into a gas intake/venting unit by means of a powered liquid transport unit, wherein the gas intake/venting unit is adapted to remove the oxygen from and replenish carbon dioxide into the culture solution, and the gas intake/venting unit comprises a first sealing member and a second sealing member, and wherein the first sealing member and the second sealing member keep the gas intake/venting unit sealed off the external environment during a process of gas intake or venting; and
introducing the photosynthesized algae and the culture solution into the pipeline photosynthesis unit cyclically.

2. The industrialized algae culturing method according to claim 1, wherein the method further comprises a step of, placing the algae and the culture solution into a cell division unit for accelerating a speed of cell division of the algae to obtain an adequate number of algae cells and increase in algae biomass before placing the algae and the culture solution into the pipeline photosynthesis unit, then the algae flow into the pipeline photosynthesis unit.

3. The industrialized algae culturing method according to claim 2, wherein the cell division unit comprises a plurality of consecutive and sealed containers, and the algae and the culture solution are placed into each of the consecutive and sealed containers respectively to allow for cell division and photosynthesis of the algae so as to produce massive algae.

4. The industrialized algae culturing method according to claim 3, wherein each of the consecutive and sealed containers is connected with a blowing device, and the blowing device is adapted to blow carbon dioxide into the consecutive and sealed container to induce flow of the massive algae so that the algae are evenly distributed in the culture solution to promote growth, cell division and increase in biomass of the algae.

5. The industrialized algae culturing method according to claim 1, wherein the method further comprises: introducing the algae and the culture solution from the gas intake/venting unit into a communicating pipe unit to physiologically regulate the algae for a period of time.

6. The industrialized algae culturing method according to claim 5, wherein, by virtue of a height difference in liquid levels between the culture solution in the gas intake/venting unit and the culture solution in the communicating pipe unit, the physiologically regulated algae and the culture solution are forced into a transparent piping cyclically.

7. The industrialized algae culturing method according to claim 1, wherein the method further comprises: introducing the algae and the culture solution from the gas intake/venting unit into a plurality of communicating pipe units that are in continuous flow communication so as to physiologically regulate the algae continuously for a period of time.

8. The industrialized algae culturing method according to claim 7, wherein each of the communicating pipe units is connected with a gas pumping device, and the gas pumping device is adapted to fill carbon dioxide into the communicating pipe unit that is in continuous flow communication to induce flow of the massive algae so that the algae are evenly distributed in the culture solution to promote growth, cell division and increase in biomass of the algae, and wherein a switching valve assembly is connected at a bottom of every two of the communicating pipe units to discharge the algae.

9. The industrialized algae culturing method according to claim 1, wherein the method further comprises: introducing the algae and the culture solution that have passed through the pipeline photosynthesis unit into a plurality of communicating pipe units that are in continuous flow communication, and then introducing the algae and the culture solution into the gas intake/venting unit by means of the powered liquid transport unit.

10. The industrialized algae culturing method according to claim 9, wherein each of the communicating pipe units is connected with a gas pumping device, in which the gas pumping device is adapted to fill carbon dioxide into the communicating pipe unit that is in continuous flow communication to induce flow of the massive algae so that the algae are evenly distributed in the culture solution to promote growth, cell division and increase in biomass of the algae, and wherein a switching valve assembly is connected at a bottom of every two of the communicating pipe units to discharge the algae.

11. An industrialized algae culturing system, comprising:
a pipeline photosynthesis unit, comprising a transparent piping that contains algae and a culture solution therein;
a powered liquid transport unit, communicating with the pipeline photosynthesis unit at the upstream thereof and pressurizing the culture solution in the transparent piping;
a gas intake/venting unit, being connected with the powered liquid transport unit at the upstream thereof and comprising a closed liquid collecting cylinder, a venting pipe and a gas feeding pipe, wherein the venting pipe keeps the closed liquid collecting cylinder sealed off the external environment by means of a first sealing member, and the gas feeding pipe keeps the closed liquid collecting cylinder sealed off the external environment by means of a second sealing member; and
a communicating pipe unit, communicating with the gas intake/venting unit at the upstream thereof and with the pipeline photosynthesis unit at the downstream thereof.

12. The industrialized algae culturing system according to claim 11, wherein the system further comprises a cell division unit connected at the upstream of the pipeline photosynthesis unit, wherein the cell division unit comprises a plurality of consecutive and sealed containers in which the algae experience cell division to produce the algae with biomass increased and the culture solution.

13. The industrialized algae culturing system according to claim 12, wherein each of the consecutive and sealed containers comprises a plurality of small-sized test tubes, a medium-sized beaker and an air-lift photosynthesis unit, and wherein the medium-sized beaker is connected at the downstream of the small-sized test tubes, the air-lift photosynthesis unit is connected at the downstream of the medium-sized beaker, and the air-lift photosynthesis unit is adapted to receive illumination from a light source module.

14. The industrialized algae culturing system according to claim 13, wherein the light source module comprises a first light source, a second light source and a power supply, and wherein the power supply is electrically connected to the first light source and the second light source, the first light source is disposed outside the air-lift photosynthesis unit and the second light source is disposed inside the air-lift photosynthesis unit.

15. The industrialized algae culturing system according to claim 13, wherein the consecutive and sealed containers are connected with a blowing device which comprises a first piping, a second piping and a third piping, and wherein the first piping is connected with each of the small-sized test tubes, the second piping is connected with each of the medium-sized beakers, and the third piping is connected with each of the air-lift photosynthesis units.

16. The industrialized algae culturing system according to claim 11, wherein the gas intake/venting unit and the communicating pipe unit are arranged side by side, the communicating pipe unit has an enlarged communicating pipe, and when a liquid level of the culture solution collected in the closed liquid collecting cylinder is higher than a height of the enlarged communicating pipe, a resulting differential pressure will force the culture solution induced into the transparent piping.

17. The industrialized algae culturing system according to claim 11, wherein the transparent piping further communicates with a ventilation valve assembly at a middle section thereof, and the ventilation valve assembly comprises a communicating water pipe connected with the transparent piping, a venting pipe connected with the communicating water pipe and having a height beyond that of the transparent piping, a switching valve for switching the communicating water pipe, and a drain valve.

18. The industrialized algae culturing system according to claim 11, wherein the closed liquid collecting cylinder of the gas intake/venting unit further communicates with at least a transparent first liquid storage pipe at the bottom, the communicating pipe unit communicates with the gas intake/venting unit at the upstream via the first liquid storage pipe, and an end of the transparent piping further communicates with a second liquid storage pipe, and wherein the second liquid storage pipe is connected with the powered liquid transport unit.

19. The industrialized algae culturing system according to claim 11, wherein the communicating pipe units are provided to form a plurality of communicating pipe units that are in continuous flow communication, and each of the communicating pipe units is connected with a gas pumping device, and wherein a switching valve assembly is connected at a bottom of every two of the communicating pipe units.

20. The industrialized algae culturing system according to claim 11, wherein an end of the transparent piping is further connected with a plurality of communicating pipe units that are in continuous flow communication, the powered liquid transport unit communicates with the communicating pipe units that are in continuous flow communication, and wherein each of the communicating pipe units that are in continuous flow communication is connected with a gas pumping unit, and wherein a switching valve assembly is connected at a bottom of every two of the communicating pipe units that are in continuous flow communication.
